(19) United States
(12) Patent Application Publication
(10) Pub. No.: US 2008/0082051 A1
(43) Pub. Date: Apr. 3, 2008

(54) DEVICE AND METHOD FOR FACILITATING INTRODUCTION OF GUIDEWIRES INTO CATHETERS

(75) Inventors: David R. Miller, Cupertino, CA (US); Michael Smith, San Jose, CA (US)

Correspondence Address: HAYNES AND BOONE, LLP, 901 Main Street, Suite 3100, Dallas, TX, 75202, US

(73) Assignee: Kyphon Inc., Sunnyvale, CA (US)

(21) Appl. No.: 11/771,768

(22) Filed: Jun. 29, 2007

Related U.S. Application Data
(60) Provisional application No. 60/826,472, filed on Sep. 21, 2006.

(51) Int. Cl. A61M 39/10

(52) U.S. Cl. 604/164.13; 604/103.04; 604/171; 606/108

(57) ABSTRACT

A guide structure for facilitating the introduction of a guidewire into a catheter comprises a body having a guide channel therethrough. An attachment end of the guide structure is adapted to receive and align a distal end of the catheter within the guide channel. A receiving end of the guide structure is adapted to receive and align a guidewire with a guidewire lumen within the catheter, when the catheter is held within the guide structure.

### CROSS-REFERENCES TO RELATED APPLICATIONS

This application claims the benefit under 35 USC 119(e) of U.S. Provisional Application No. 60/826,472 filed on Sep. 21, 2006, the full disclosure of which is incorporated herein by reference.

### BACKGROUND OF THE INVENTION

1. Field of the Invention

The present invention is related generally to medical apparatus and methods. In particular, the invention is directed at structures and methods for aligning and introducing guidewires into guidewire lumens in medical catheters.

A wide variety of medical procedures require placing a catheter or other medical device over a guidewire. Many of these procedures are performed in the vasculature, including angioplasty, atherectomy, stent delivery, and the like. Many other procedures are performed in non-vascular lumens, including urinary procedures in the urethra and ureter, and the like. Of particular interest to the present invention are those procedures performed with very small diameter catheters which must be introduced over very small guidewires, such as the Functional Anesthetic Discography (FAD), a procedure in the spinal disc space, which utilizes a guidewire, which utilizes a guidewire typically having a diameter of about 0.009 inches, and a diameter of a guidewire lumen in a catheter of about 0.011 inches. The guidewires are placed under sterile conditions in the operative environment where introduction of guidewires into the catheter can be difficult, particularly when very small catheters and guidewires are involved.

For these reasons, it would be desirable to provide apparatus and methods for facilitating the introduction of an end of a guidewire into the port of a guidewire lumen in a medical catheter. It would be particularly desirable if such methods and apparatus assured the proper alignment of the guidewire with minimum effort, even under conditions of low light and limited visibility, as often is the case in radiology laboratories where many of these procedures are performed.

2. Description of Background Art

U.S. Patent Publication No. 2005/0234425 describes a FAD catheter which may be introduced over a guidewire and which can benefit from the facilitated guidewire introduction apparatus and methods of the present invention.

### BRIEF SUMMARY OF THE INVENTION

The present invention provides apparatus and methods for facilitating the introduction of a guidewire into a guidewire lumen in a medical catheter. The invention may be particularly useful for placement of very small guidewires, which may have, for example, diameters of about 0.009 inches or below, into very small guidewire lumens, which may have, for example, diameters of about 0.011 inches or below.

In one variation, the apparatus of the present invention comprises guidewire structures for loading a guidewire into the lumen of a catheter. The guidewire structures comprise a body (also referred to as “guide-body”) having a receiving end, an attachment end, and a guide channel therebetween. The attachment end removably attaches to an end of the guidewire such that the guidewire lumen of a catheter is aligned with the guide channel. The receiving end of the guide structure body receives an end of the guidewire such that the guidewire can be advanced through the guide channel and into the lumen of the catheter. Typically, the guide structure may further comprise a handle on the body to facilitate manipulation during the guidewire introduction. The guidewire may be conveniently located to project laterally from a location at or near the attachment end of the body, although it could also be at other locations.

In the specific embodiments, the attachment end of the body of the guide structure is flared to provide an enlarged target area for capturing the catheter and guiding the catheter into the guide channel. Thus, in one aspect, a catheter may be inserted into the guide structure at the attachment end of the guide body and through the guide channel. In another aspect, a portion of the guide channel may be shaped to conform to the shape of an outer distal end of the catheter to, for example, form a tapered section or, a “stop” or “limiting stop” that engages and aligns the lumen of the catheter with the guide channel. Thus, when the catheter is introduced into the attachment end of the guide body, the catheter will fit snugly so that it is aligned within the guide channel and can be inserted to a preselected depth as determined by the limiting stop.

In other exemplary aspects of the guide structure, the receiving end of the guide body may also be flared to provide an enlarged target area for receiving the guidewire. Often, the flare is generally conical in shape and coaxially positioned around an axis of the guide channel. A transition region may be provided in the guide channel for aligning the guidewire as it is advanced through the guide channel at the open end or port of the guidewire lumen in the catheter. Thus, after placing the catheter into the attachment end of the guide structure, the physician may easily advance the guidewire through the receiving end and into the guidewire lumen of the catheter.

In another specific aspect of the present invention, the guide body may be adapted to laterally release the catheter and guidewire from the guide structure or remove the guide structure from the catheter and guidewire assembly. For example, the guide body may be axially slit or splittable along a wall of the guide channel. A slit would permit the catheter and guidewire to be pulled from the channel while leaving the guide body intact. Splittable guide bodies will allow the user to pull the guide body apart and remove the guide structure from the catheter and guidewire. In other embodiments, the guide body may be open along one side of the guide channel so that the catheter and guidewire may be pulled from the channel with minimum effort. With such open channel configurations, detents, protrusions, or other simple components may be provided to hold the catheter in place as the guidewire is advanced.

Methods according to the present invention for inserting a guidewire into the lumen of a catheter comprise removably attaching a guide structure onto an end of the catheter. The guide structure includes a channel which aligns with a guidewire port on the lumen when the catheter is in place in the channel. After the catheter has been placed in the guide structure, an end of the guidewire is introduced through the channel of the guidewire structure and into the lumen of the catheter. After the guidewire is in place in the catheter, the guide structure may be removed from the catheter.

Removably attaching the guide structure to the catheter may comprise inserting an end of the catheter through a flared attachment end of the guide structure channel. Typically, when introduced through the flared attachment end of the guide structure, the catheter will be captured in a shaped portion of the channel where it engages a stop for final positioning.

Introducing an end of the catheter typically comprises inserting the guidewire end through a flared receiving end of the guide structure channel after the guide structure has been attached to the catheter. Typically, the flared receiving end has a generally conical shape.

Removing the guide structure from the catheter may comprise pulling the catheter and guidewire laterally from the guide structure. In such cases, the guidewire and catheter may be pulled through an axial slit which is preformed in the guide structure. Alternatively, the guidewire and catheter may be pulled through a pre-formed discrete opening in the channel. Alternatively, the guide structure may be removed from the catheter and guidewire by splitting the guide structure, typically by pulling the guide structure apart at one end so that the structure separates into two pieces which may then be pulled away from the catheter and guidewire. Alternatively the guide structure could be radially expanded facilitating removal from the catheter by passing it over the proximal end of the catheter.

In yet another variation, the guide structure is utilized for placement of a catheter over the proximal portion of an elongated and flexible guidewire having its distal end inserted within a patient's body. In one example, a physician first inserts the distal portion of a flexible and elongated guidewire into a patient's body through one of various methods that are well known to one of ordinary skill in the art. The physician then places a guide structure over the distal portion of a catheter. The distal widened-end (e.g., funnel-shaped opening) of the guide structure is then placed over the proximal end of the flexible guidewire. With the guide structure directing the distal tip of guidewire, the catheter is advanced over the proximal portion of the guidewire. Once the catheter is placed over a portion of the guidewire, the physician may then remove the guide structure. The physician can further propel the catheter along the length of the guidewire until the distal portion of the catheter enters the patients body.

In one application, the guide structure is preloaded on the catheter, and provided to the physician in a sterile package, such that the physician may place the catheter onto the guidewire right out of the package without the need to further position and/or orient the guide structure. In addition, the catheter may have a penetrating tip (e.g., a stiffened and/or tapered tip portion) and/or an anchoring mechanism (e.g., an expandable balloon) at the distal tip portion. The guide structure may be configured to be preloaded on the distal portion of the catheter to provide protection for the penetrating tip and/or the anchoring mechanism. In one variation, the inner lumen of the guide structure is configured with a profile for receiving the penetrating tip and/or the anchoring mechanism.

In another aspect, embodiments of the present invention provide an apparatus. The apparatus comprises a guide structure, and a catheter.

In many embodiments, the apparatus comprises a sterile package with the guide structure pre-loaded on the catheter within a sterile package. The catheter may be on the catheter within the sterile package such that a user may place the catheter onto a guidewire. The catheter can be positioned within a guide channel of the guide structure, and the guide structure can be removably attached to the catheter. In some embodiments, the catheter comprises a balloon and a penetrating tip. The guide structure can be configured to protect the penetrating tip of the catheter and the balloon of the catheter.

In many embodiments, the apparatus comprises a guidewire. The guidewire may comprise a diameter of about 0.009 inches or below. The catheter may comprise a lumen with a diameter of about 0.011 inches or below.

In many embodiments, the guide structure comprises a guide body having a receiving end, an attachment end, and a guide channel extending therebetween. The receiving end may comprise a diameter of about 0.25 inches. In some embodiments, the receiving end comprises a diameter in a range from about 0.20 to about 0.27 inches. The guide channel may comprise a diameter within a range from about 0.035 to about 0.038 inches. The guide channel diameter may be sized from about 0.0001 inches to about 0.010 inches smaller than a diameter of the catheter so as to form an interference fit. The guide channel comprises a guidewire section with a diameter within a range from about 0.010 inches to about 0.014 inches.

In specific embodiments, the attachment end may comprises a diameter from about 0.035 inches to about 0.05 inches.

It is also contemplated that the guide structure and method of use thereof, may be implemented in combination with the devices and methods described in U.S. Patent Publication No. 2005/0234425, which is incorporated herein by reference in its entirety for all purposes. In one example, the method comprises placing the distal portion of an elongated (e.g., 12 inches or longer) and flexible guidewire percutaneously into a spinal disc in a patient's body. The distal widened end of a guide structure, which is attached to the distal portion of a catheter, is placed over the distal tip of the guidewire. As the physician advance the catheter forward in relation to the guidewire, a transition region within the guide structure aligns the guidewire with the lumen of the catheter. Once the distal portion of the catheter is inserted over the proximal portion of the guidewire, the physician may then remove the guide structure through various methods described herein. For example, the catheter can be advanced distally such that the guide structure contacts an introducer device, for example an introducer needle, and the guide structure is released from the end of the catheter. The distal portion of the catheter is further advanced over the guidewire and into the patients body, followed by penetration into the spinal disc. Once the distal portion of the catheter has entered the disc, an optional anchoring mechanism on the catheter may be deployed to secure the distal portion of the catheter within the disc.

### BRIEF DESCRIPTION OF THE DRAWINGS

FIG. 1 illustrates one embodiment of the guide structure constructed in accordance with the principles of the present invention.

FIGS. 2A-2D illustrate the steps in introducing a guidewire into a catheter using the exemplary guide structure of FIG. 1.

FIG. 3 illustrates the guide structure used with an FAD catheter comprising an anchor, according to embodiments of the present invention.

FIG. 4 illustrates dimensions of the guide structure, according to embodiments of the present invention.

### DETAILED DESCRIPTION OF THE INVENTION

The present invention provides apparatus and methods for facilitating the introduction of a guidewire into a guidewire lumen in a medical catheter. Embodiments of the invention may be particularly useful for placement of very small guidewires, which may have, for example, diameters of about 0.009 inches or below, into very small guidewire lumens, which may have, for example, diameters of about 0.011 inches or below. The apparatus may comprise guidewire structures for loading a guidewire into the lumen of a catheter. The guidewire structures comprise a body having a receiving end, an attachment end, and a guide channel therebetween. The attachment end removably attaches to an end of the guidewire such that the guidewire lumen of a catheter is aligned with the guide channel. The receiving end of the guide structure body receives an end of the guidewire such that the guidewire can be advanced through the guide channel and into the lumen of the catheter. Typically, the guide structure may further comprise a handle on the body to facilitate manipulation during the guidewire introduction. The guidewire may be conveniently located to project laterally from a location at or near the attachment end of the body, although it could also be at other locations.

The flared receiving end of the guide structure channel typically has an opening or “target area” that is substantially larger than the lumen in the catheter, often having a diameter of about 5 to about 1000 times larger than the guidewire diameter. In one variation, the opening flare or funnel is configured with a diameter of about 0.25 inches and tapers down to an area about the size of the lumen in the catheter that accepts the guidewire. The tapered down diameter of the flare is the same size or slightly larger than the guidewire and preferably the same size or slightly smaller than the lumen in the catheter that accepts the guidewire. The included angle of the interior guidewire flare or funnel could be between about 5 and about 120 degrees.

In some aspects, the catheter may be back-loaded over a guidewire that is already in place in the patient, and the guide structure may be trapped at the distal end of the catheter after the catheter has been place over the guidewire. Thus, the guide structure may be adapted to be removed from the guidewire and catheter assembly. For example, the guide structure may be (a) adapted for splitting or peeling the guide structure longitudinally into two or more parts or (b) have an axial slit or opening such that the guidewire and catheter assembly can be pulled through the slit or opening. Such slit in the channel may be, for example, a substantially straight slit, a spiral cut, or a combination of a straight slit or spiral cut. The slit, for example, could also be complete or intermittent (also referred to as perforated). Such a perforated slit would allow the guide structure to function like an intact tube, but still allow removal of the guide structure from the catheter and guidewire assembly by splitting the slit.

In another aspect, the guide structure may have one or more pull tabs that assist in its removal from the catheter and guidewire assembly, particularly by splitting. Such pull tabs may be located on the guide structure and may be sized such that the pull tabs may be easily grasped.

Other optional features of the guide structure include an interior tapered section or “stop” that engages and aligns the lumen of the catheter with the channel. This tapered section allows for proper alignment between the guide structure and the catheter for catheters with lumens that are not in the middle of the device and for variations in size and construction of the catheter or guide structure. The included angle of the interior tapered section could be between about 5 and about 120 degrees. In a preferred aspect, the angle of the interior tapered section could be about 60 degrees.

In another embodiment, the guide structure may be secured to the catheter by an interference fit (also known as press fit or friction fit) such that after the guidewire is introduced into the lumen of the catheter the guide structure and catheter are securely held together by the friction between two components, yet the guide structure and catheter may be are easily separable by use of moderate force. The interference fit may be formed by sizing the guide channel to have an inside diameter of about 0.0001″ to 0.010″ smaller than the outside diameter of the catheter. An interference fit may also be formed by small ribs in the channel or may be formed by different shapes for the channel and catheter (for example one straight, one curved, or one straight, one corkscrew shaped).

In another embodiment, the catheter may have a balloon near its distal tip. The balloons may require having a sleeve or cover over them during storage and shipment which may, for example, protect the balloon from damage and/or keep the balloon pleats compressed in order to minimize the passing profile of the balloon. In a particular embodiment, the guide structure is integrated into a balloon cover. Examples of balloons useful in embodiments of the present invention are described in U.S. Pub. No. 2005/0234425, the full disclosure of which has been previously incorporated herein by reference.

Various materials may be used to make the guide structure as required of a specific application. The guide structure, for example, may be made out of a soft material such as a polymer extrusion. Polymer extrusion materials that may be used include, but are not limited to nylon 11, nylon 12, high-density polyethylene, low-density polyethylene, PEEK, urethane, and polyurethane. The guide structure could also be injection molded. In a preferred embodiment the guide structure is formed out of extruded high-density polyethylene.

In another embodiment, the material used to make the guide structure may comprise or contain a material that is radiopaque. A radiopaque material may allow the physician to visualize the guide structure using fluoroscopy.

In a further embodiment, the material of the guide structure could consist of or contain a material that may glow or ruminate in the dark or under light or visibility. This would allow the physician to more easily place, locate, and manipulate the structure in a dark room or room which is under low light or limited visibility.

In yet a further embodiment, the material used to make the guide structure may comprise or contain, or be coated with a material that is highly lubricious. Such a material would allow for (a) easier assembly of the guide structure onto the catheter, (b) easier removal of the guide structure from the catheter, or (c) easier passage of the guidewire through the guide structure and into the catheter. Such material may be, for example, a hydrophilic or hydrophobic coating, a dry lubricant like parylene, or a material from the Teflon family such as PTFE, ePTFE, or FEP.

A guide structure may be manufactured in a variety of different ways. For example, the guide structure may be formed from an extruded tube, by using methods such as heating and using specialized tooling to create the desired interior and exterior features, or the guide structure may be injection molded or machined into its final form. A slit in the guide structure or in a portion of the guide structure may be created using a variety of techniques known in the art such as using a laser, using a heated element, or using a cutting tool. The resulting slit may be about 0.0001″ to about 0.050″ in width. Alternatively the guide structure may be biased to close after the slit is formed which may result in a slit width of zero width unless the slit is forcibly opened. Alternatively the slit may have a width that varies over its length.

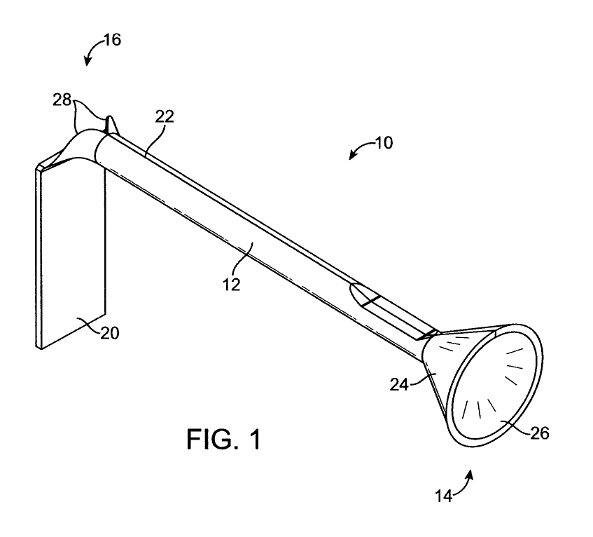

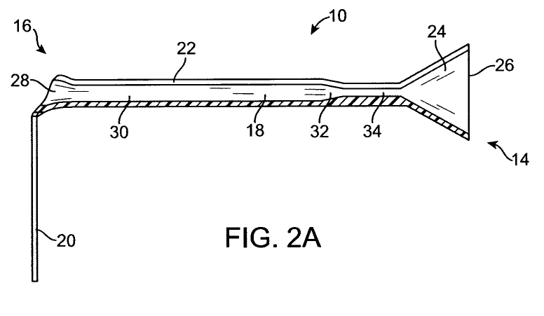

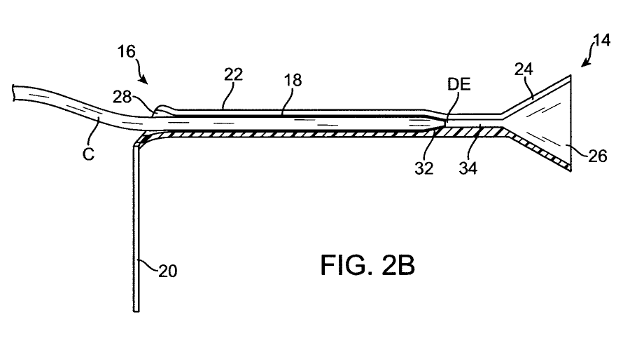

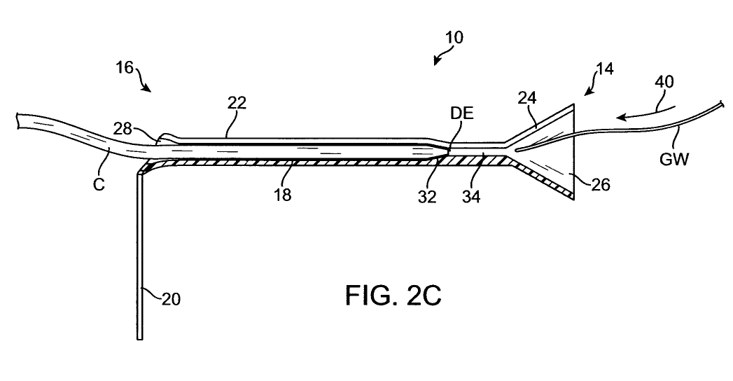

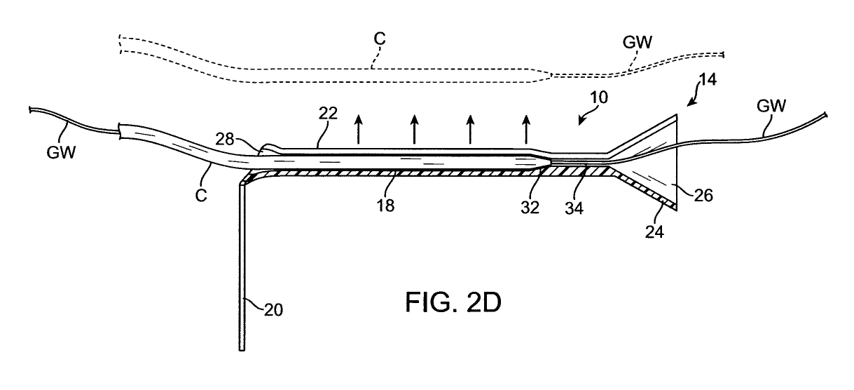

Referring now to FIG. 1, an exemplary embodiment of a guide structure constructed in accordance with the principles of the present invention will be described. The guide structure 10 comprises a body 12 having a receiving end 14 and an attachment end 16. A guide channel 18 (best seen in FIG. 2A) extends between the receiving end 14 and attachment end 16. A handle 20 may be provided, typically projecting laterally from near the attachment end of the guide structure. In the illustrated embodiment, an axial slit 22 extends the entire length of the guide structure 10 so that the assembly of the catheter and guidewire may be pulled from the guide structure after the loading procedure is completed.

The receiving end 14 of the guide structure is typically formed as a conical extension or funnel 24 having an open end or target area 26 through which the physician inserts a guidewire into the catheter. Similarly, the attachment end 16 of the guide structure may be flared, for example having a pair of diverging sections 28 which help channel the catheter into the channel. Generally, as the catheter body is larger and easier to manipulate than the guidewire, the amount of flaring on the attachment end 16 of the guide structure 10 need not be as great or as large as the diameter of the receiving end 14.

Referring now to FIGS. 2A-2D, a catheter C (FIG. 2B) is introduced through the flared section 28 of the guide structure 10 and is received in a section 30 of channel 18 which is configured to conform closely to the distal end of the catheter. A stop section 32 is formed in the channel, where the stop is typically tapered to conform to the distal end DE of the catheter, as shown in FIG. 2B. The dimensions and alignments of the conforming section 30 and stop 32 are selected so that a distal guidewire port (not shown) in catheter C will be aligned with guidewire section 34 of the channel 18.

Thus, once the catheter C is in place in the guide channel 18, as shown in FIG. 2B, the guidewire GW is inserted through the target area 26 of the conical structure 24 in the direction of arrow 40, as shown in FIG. 2C. As the end of the guidewire GW is advanced, it is directed into the guidewire section 34 of channel 18, as shown in FIG. 2D. Once in the distal end of the guidewire lumen in catheter C, the guidewire GW may be further or fully advanced into the catheter. After such advancement, it will usually be necessary to remove the guide structure 10 from the assembly of the catheter C and guidewire GW. In the illustrated embodiment, this can be achieved by pulling the catheter and guidewire through the axial slit 22 in the direction of the arrows shown in FIG. 2D. Alternatively, as discussed elsewhere, the guide structure could be axially split, or the slit could be spiral or have other configurations. Alternatively, the guide structure could be removed by passing the guide structure proximally off of the catheter by pulling tab 20 or pushing in a proximal direction on the target area 26 or any portion of the guide catheter. Once the guide structure 10 has been removed, the catheter may be advanced into the patient over the guidewire in a conventional manner.

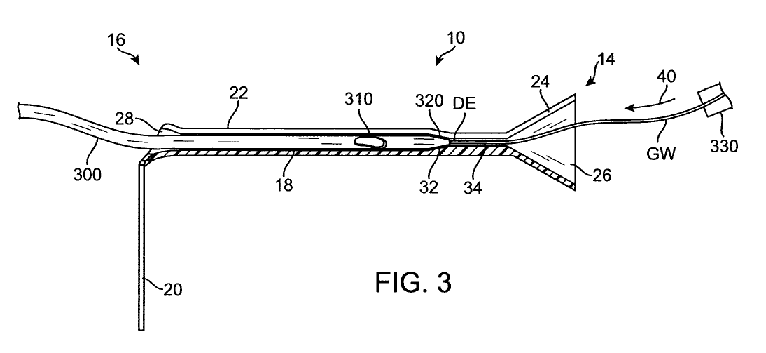

FIG. 3 illustrates the guide structure used with an FAD catheter 300 comprising an anchoring mechanism 310, according to embodiments of the present invention. The guide structure as described above is shown with an FAD catheter, as describe in U.S. Patent Publication No. 2005/0234425, the full disclosure of which has been previously incorporated herein by reference. Catheter 300 may comprise a penetrating tip 320. The guide structure can preloaded on the catheter, and provided to the physician in a sterile package, such that the physician may place the catheter onto the guidewire right out of the package without the need to further position and/or orient the guide structure. In addition, penetrating tip 320 may comprise, a stiffened and/or tapered tip portion. Anchoring mechanism 310 may comprise an expandable balloon at the distal tip portion. The guide structure may be configured to be preloaded on the distal portion of the catheter to provide protection for the penetrating tip and/or the anchoring mechanism. In some embodiments, the inner lumen of the guide structure is configured with a profile for receiving the penetrating tip and/or the anchoring mechanism.

In some embodiments, guide structure may be removed by advancing the catheter distally such that the guide structure contacts an introducer device 330, or introducer needle, as described in U.S. Patent Publication No. 2005/0234425, previously incorporated herein by reference. Contact between the guide structure and introducer device 330 can split open the slit in the guide structure so as to release the guide structure from the distal end of the catheter, for example by falling off the distal end of the catheter.

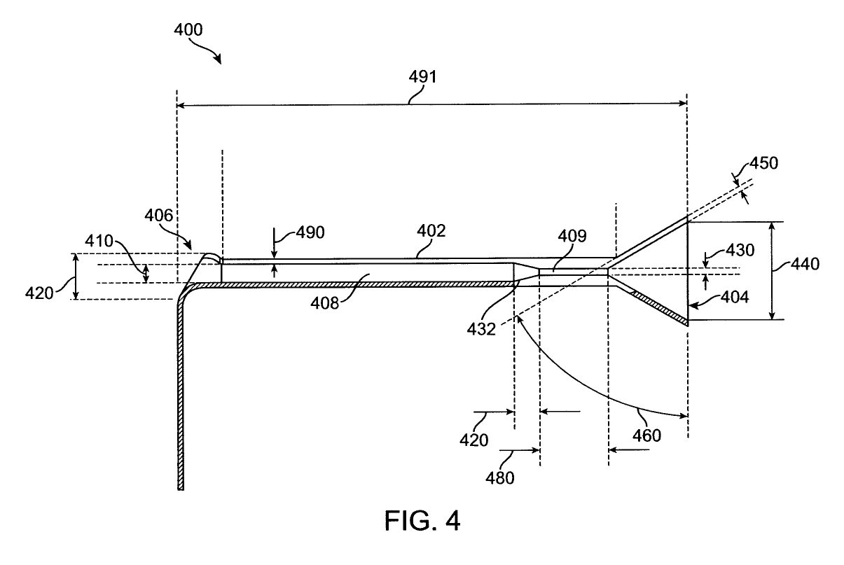

FIG. 4 illustrates dimensions of the guide structure 400, according to embodiments of the present invention. The guide structure comprises a guide body 402 having a receiving end 404, an attachment end 406, and a guide channel 408 extending therebetween. The receiving end comprises a diameter 440 within a range from about 0.20 to about 0.27 inches, for example about 0.25 inches. The guide channel comprises a diameter 410 within a range from about 0.035 to about 0.038 inches. The guide channel diameter is sized from about 0.0001 inches to about 0.010 inches smaller than a diameter of FAD catheter 300 so as to form an interference fit. The guide channel comprises a guidewire section 409 with a diameter 430 within a range from about 0.010 inches to about 0.014 inches. Attachment end 406 comprises a diameter 420 within a range from about 0.035 inches to about 0.05 inches. Receiving end 404 extends for the body at an angle 460 within a range from about 55 to 65 degrees, for example about 60 degrees. A length 420 of a stop section 432 comprises a length of about 0.04 inches. Guidewire section 409 comprises a length 480 of about 0.14 inches. Receiving end 404 comprises a thickness 450 of about 0.12 inches. A thickness of the body wall is about 0.01 inches. An overall length 491 of guide structure 400 may be within a range from about 0.9 to about 1.1 inches, for example about 1 inch.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A guide structure for leading a guidewire into a lumen of a catheter, said guide structure comprising:
a body having a receiving end, an attachment end, and a guide channel therebetween;
wherein the attachment end removably attaches an end of the catheter such that the catheter lumen is aligned with the guide channel;
wherein the receiving end receives an end of the guidewire such that the guidewire can be advanced through the channel and into the lumen of the catheter.

2. A guide structure as in claim 1, further comprising a handle on the body.

3. A guide structure as in claim 2, wherein the handle projects laterally from a location at or near the attachment end of the body.

4. A guide structure as in claim 1, wherein the attachment end of the body is flared to provide an enlarged area for capturing the catheter.

5. A guide structure as in claim 1, wherein a portion of the guide channel is shaped to conform to an outer distal end of the catheter.

6. A guide structure as in claim 5, wherein the shaped portion of the guide body channel has a stop for limiting insertion of the catheter.

7. A guide structure as in claim 1, wherein the receiving end of the body is flared to provide an enlarged area for receiving the guidewire.

8. A guide structure as in claim 7, wherein the flared receiving end is generally conical.

9. A guide structure as in claim 1, wherein the body is adapted to laterally release the guidewire and/or catheter.

10. A guide structure as in claim 9, wherein the guide body channel is axially slit or splittable.

11. A guide structure as in claim 9, wherein the guide body channel is at least partially open.

12. A method for loading a guidewire into a lumen of a catheter, said method comprising:
removably attaching a guide structure onto an end of the catheter, wherein the guide structure includes a channel which aligns with a port on the lumen;
introducing an end of the guidewire through the channel of the guide structure into the lumen of the catheter; and
removing the guide structure from the catheter.

13. A method as in claim 12, wherein removably attaching comprises inserting the catheter end through a flared attachment end of the guide structure channel.

14. A method as in claim 13, wherein the catheter end is captured in a shaped portion of the channel and engages a stop for final positioning.

15. A method as in claim 12, wherein introducing an end of the guidewire comprises inserting the guidewire end through a flared receiving end of the guide structure channel after the guide structure has been attached to the catheter.

16. A method as in claim 15, wherein the flared receiving end has a generally conical shape.

17. A method as in claim 12, wherein removing comprises pulling the catheter and guidewire laterally from the guide structure.

18. A method as in claim 17, wherein the guidewire and catheter are pulled through an axial slit in the guide structure.

19. A method as in claim 17, wherein the guidewire and catheter are pulled through an opening in the channel.

20. A method as in claim 12, wherein removing comprises splitting the guide structure from the catheter and guidewire

21. An apparatus comprising:
a guide structure; and
a catheter.

22. The apparatus of claim 21 further comprising a sterile package with the guide structure pre-loaded on the catheter within a sterile package.

23. The apparatus of claim 22 wherein the guide structure is on the catheter within the sterile package such that a user may place the catheter onto a guidewire.

24. The apparatus of claim 23 wherein the catheter is positioned within a guide channel of the guide structure, and the guide structure is removably attached to the catheter.

25. The apparatus of claim 22 wherein the catheter comprises a balloon.

26. The apparatus of claim 22 wherein the catheter comprises a penetrating tip.

27. The apparatus of claim 22 wherein the guide structure is configured to protect a penetrating tip of the catheter and a balloon of the catheter.

28. The apparatus of claim 21 further comprising a guidewire.

29. The apparatus of claim 28 wherein the guidewire comprises a diameter of about 0.009 inches or below.

30. The apparatus of claim 29 wherein the catheter comprises a lumen with a diameter of about 0.011 inches or below.

31. The apparatus of claim 21 wherein the guide structure comprises a guide body having a receiving end, an attachment end, and a guide channel extending therebetween.

32. The apparatus of claim 31 wherein the receiving end comprises a diameter of about 0.25 inches.

33. The apparatus of claim 31 wherein the receiving end comprises a diameter within a range from about 0.20 to about 0.27 inches.

34. The apparatus of claim 31 wherein the guide channel comprises a diameter within a range from about 0.035 to about 0.038 inches.

35. The apparatus of claim 34 wherein the guide channel diameter is sized from about 0.0001 inches to about 0.010 inches smaller than a diameter of the catheter so as to form an interference fit.

36. The apparatus of claim 31 wherein the guide channel comprises a guidewire section with a diameter within a range from about 0.010 inches to about 0.014 inches.

37. The apparatus of claim 31 wherein the attachment end comprises a diameter within a range from about 0.035 inches to about 0.05 inches.
